(19) United States
(12) Patent Application Publication
(10) Pub. No.: US 2026/0248400 A1
(43) Pub. Date: Aug. 27, 2026

(54) PHOTONIC INTEGRATED CIRCUIT

(71) Applicant: ROCKLEY PHOTONICS LIMITED, Altrincham (GB)

(72) Inventors: Craig GARDNER, Belmont, MA (US); Jeffrey DRISCOLL, San Jose, CA (US); Kate LeeAnn BECHTEL, Pleasant Hill, CA (US)

(21) Appl. No.: 19/650,135

(22) Filed: Apr. 16, 2026

Related U.S. Application Data
(63) Continuation of application No. 17/814,787, filed on Jul. 25, 2022, now abandoned.
(60) Provisional application No. 63/227,227, filed on Jul. 29, 2021.

(51) Int. Cl. A61B 5/026; A61B 5/00; A61B 5/02

(52) U.S. Cl. CPC A61B 5/0261; A61B 5/0077; A61B 5/02007

(57) ABSTRACT

A device for investigating blood flow at a portion of tissue of a user includes a laser configured to generate light for transmission to the portion of the tissue; a plurality of detectors; and a plurality of separate optical inputs respectively optically coupled to the plurality of detectors and configured to respectively receive a plurality of separate speckle patterns, responsive to the transmitted light, from the portion of the tissue, wherein the device is configured to obtain a speckleplethysmography (SPG) measurement based on a plurality of measurements taken respectively by the plurality of detectors.

### CROSS-REFERENCE TO RELATED APPLICATION(S)

This application is a continuation of U.S. patent application Ser. No. 17/814,787, filed Jul. 25, 2022, which claims priority to and the benefit of U.S. Provisional Application No. 63/227,227, filed Jul. 29, 2021, entitled “PHOTONIC INTEGRATED CIRCUIT”; the entire contents of all of the documents identified in this paragraph are incorporated herein by reference.

### FIELD

One or more aspects of embodiments according to the present invention relate to Photonic Integrated Circuits/Chips (PICs), and more particularly to PICs for use in speckleplethysmography (SPG).

### BACKGROUND

It is known that speckle patterns taken at tissue can be compared to observe biomedical parameters such as blood flow. Examples of such systems can be seen at U.S. Pat. No. 10,357,165 (Samsung); U.S. Pat. No. 10,568,527 (Samsung), U.S. Pat. No. 11,045,103 (Samsung), U.S. Pat. No. 10,750,956 (ContinUse Biometrics Ltd), and U.S. Pat. No. 9,848,787 (White et al.).

Speckleplethysmography is a technique giving blood flow index as a function of time.

Photoplethysmography is a technique giving blood volume as a function of time.

Wearable devices on the market typically use PPG signal analysis (with or without ECG) to extract blood pressure. Very few wearables point to use of speckle. Those that do, often measure changes in speckle pattern.

### SUMMARY

Accordingly, the present invention aims to solve the above problems by providing, according to a first aspect, a photonic integrated device comprising: a photonic integrated circuit (PIC) adapted to investigate blood flow at a portion of tissue of a user, said PIC comprising: a laser having an optical output, or waveguide for guiding an optical output from an external laser, the optical output being split into a first optical component and a second optical component; wherein the first optical component is arranged to be transmitted to and generate speckle at the portion of tissue of the user; the photonic integrated device further comprising: one or more detectors, each detector configured to receive the speckle generated by the first optical component at the portion of tissue; and one or more optical splitters optically coupling the second optical component to one or more respective input(s) of the one or more detectors; wherein the photonic integrated device is further adapted to measure interference at the one or more detectors between a sample arm formed by the first optical component and a reference arm formed by the second optical component.

In this way, a device may be provided capable of measuring blood pressure via SPG measurement using an autocorrelation (interferometric) technique in a wearable form factor using a PIC that may include the laser and detector, with splitter and on-chip waveguide for coherent detection. This enables a low-cost photodiode to be used. Also, temporal autocorrelation may be performed in a custom application-specific integrated circuit (ASIC) or field programmable gate array (FPGA) that may be part of the full hardware stack that may be located on the PIC.

Typically, the laser is located on the PIC. Alternatively, a laser may be located off-chip, external to the PIC, in which case the PIC comprises a waveguide that can be coupled to the optical output of the off-chip laser in order to guide the optical output of the laser within the PIC.

The present invention also aims to solve the above problems by providing, according to a second aspect, a photonic integrated device for speckleplethysmography (SPG) measurements, comprising one laser and multiple detectors.

The present invention also aims to solve the above problems by providing, according to a third aspect, a photonic integrated device for diffuse correlation spectroscopy (DCS).

The present invention also aims to solve the above problems by providing, according to a fourth aspect, a wearable device comprising a photonic integrated device, and/or a PIC according to any embodiment described herein.

Optional features of the invention will now be set out. These are applicable singly or in any combination with any aspect of the invention.

Optionally, the device comprises an ASIC or FPGA configured to calculate a temporal autocorrelation of a detected intensity. The ASIC or FPGA may be configured to control a time lag between the first component and the second component, thereby controlling the autocorrelation. The interference occurring at the detector may be spatial or temporal interference. Temporal autocorrelation may be carried out at the detector between the sample arm formed by the first component and a reference arm formed by the second component.

Optionally, light generated by the laser is collected from the tissue portion and delivered to a respective one of the one or more detectors by a single mode waveguide and/or an aperture.

Optionally, light is collected from the tissue portion and delivered to a respective one of the one or more detectors by a multimode waveguide and/or an aperture.

Optionally, each detector receives only light from a single grain of speckle within a larger speckle pattern.

Optionally, the PIC is a silicon photonics chip.

Optionally, the laser has a wavelength of operation of 1280 nm or more.

Optionally, the PIC comprises one or more additional lasers.

The one or more detectors may be located on a physically separate chip to the laser. The timing may be controlled for detector sample acquisition. The spatial separation between the one or more detectors and the laser may be fixed.

The PIC may include a plurality of lasers. For example, two lasers may be used for calculating optical properties (e.g. an absorption coefficient for molecules such as hemoglobin).

In one or more embodiments, the one or more detectors are located on the same PIC as the laser. However, in other embodiments, the one or more detectors may be located separately from the PIC. In this case, the detectors may be located on a second PIC, or may be bonded directly to a printed circuit board and arranged to collect the light from speckle at the tissue directly. It is envisaged that the one or more lasers may be located off chip (i.e. not directly located on the PIC, but instead optically coupled to an optical input of the PIC).

In some embodiments, the laser is a fixed wavelength laser. Alternatively, in some embodiments, the laser is a tunable laser. The PIC may comprise multiple lasers, and the multiple lasers may comprise fixed wavelength lasers, tunable lasers, or a mixture of both. Where multiple lasers are present, the photonic integrated device is typically configured, for example by a switching mechanism, to operate one laser at a time.

There can be advantages to operating one laser at a time. For example, peak power consumption, safety limits, and the ability to use a single direct-detect PD. One or more multiplexers may be present to multiplex multiple laser outputs.

In embodiments comprising multiple lasers, all lasers may be operated at once if there is a wavelength demultiplexer located before the detectors. A demultiplexer such as an Echelle grating before the PDs would enable the PDs to detect all wavelengths in a single shot. The system starts to grow in complexity if there are many wavelengths and many PDs per wavelength, but the principles of operation remain the same.

Further optional features of the invention are set out below.

### BRIEF DESCRIPTION OF THE DRAWINGS

Embodiments of the invention will now be described by way of example with reference to the accompanying drawings in which:

FIG. 1 is a PIC according to an embodiment of the present invention;

FIG. 2 is a PIC according to an embodiment of the present invention;

FIG. 3 is a PIC according to an embodiment of the present invention;

FIG. 4 is a PIC according to an embodiment of the present invention;

FIG. 5 is a PIC according to an embodiment of the present invention;

FIG. 6 is a schematic diagram of a PIC according to an embodiment of the present invention in use;

FIG. 7 is a schematic diagram of a PIC according to an embodiment of the present invention in use; and

FIG. 8 is an example of a method of use of a PIC according to an embodiment of the present invention.

### DETAILED DESCRIPTION

The detailed description set forth below in connection with the appended drawings is intended as a description of exemplary embodiments of a device provided in accordance with the present invention and is not intended to represent the only forms in which the present invention may be constructed or utilized.

As shown in the Figures, a PIC according to embodiments of the invention may comprise one laser and N detectors. The laser is output from the PIC to the sample. Light exiting the sample (or reflecting off surface) is input back to the PIC detectors. For some embodiments, SPG measurements are carried out and each detector is configured to measure one speckle grain or approximately one speckle grain (e.g. less than 5 speckle grains, preferably less than two speckle grains, where each speckle grain may be an interference node; for example a speckle grain may be a bright spot on a speckle pattern (a region of constructive interference) or a speckle grain may be a dark spot on a speckle pattern (a region of destructive interference). By using single mode collection means such as a single mode fiber it is possible to ensure that light from a single speckle grain is collected, regardless of where the collection means is placed. Alternatively, “few mode” or multimode fibers or waveguides may be used to collect more than one speckle grain. Few mode may correspond to 25 modes or less. While combining information from multiple speckle reduces effective signal through reduction in coherence, the signal may still be above the noise or background.

Light from sample can reach the detectors directly through apertures or via waveguides. Aperture size or waveguides can be multimode, “few mode”, or single mode. If multimode, light from waveguide may be dispersed onto multiple detectors to still get approximately one speckle (i.e. speckle grain) per detector/pixel or may be collected onto a single detector for an increase in intensity, but with reduction in effective signal as previously described.

Some of the light from the laser can be split off in the PIC and sent to each of the single mode waveguides going from sample to detectors. This enables optical heterodyne (coherent) detection, sometimes known as interferometric detection. This is important for SPG because the signal level from a single mode fiber can be very low. An additional device such as an optical combiner is typically present to combine the light from the laser and the light reflected from the skin into a single waveguide. A combiner could, for example, take the form of a 3 dB coupler (e.g. a 2×1 Y-junction or 2×1 MMI).

Detectors can collect light from a single small region or can be spaced and clustered to collect light from several small regions. Latter enables SPG waveforms from multiple regions of the sample surface.

Detector signals can be processed one at a time and then averaged across N detectors for temporal SPG. Or detector signals from a single region can be processed together for spatial SPG.

### Wavelength (Temporal Autocorrelation):

Speckle can be measured at any wavelength. For measurements in tissue, t green, yellow, red, NIR I, NIR II, NIR III/SWIR I and SWIR II bands may be utilized, where wavelengths in each band refer to known bands as exemplified in scientific papers and texts in the art.

### Mode of Operation (Temporal Autocorrelation):

Continuous wave or pulsed. For pulsed operation, the ideal pulse duration should be either significantly longer than the speckle temporal decorrelation time or significantly shorter than the speckle decorrelation time.

### Data Sampling Rate (Temporal Autocorrelation):

For speckle temporal autocorrelation, data collection should be at least 100 kHz, at least 500 kHz, typically 1 MHz and up to 10 MHz. Faster sampling is acceptable although data will likely be averaged leading into temporal autocorrelation calculation.

### Light Source Requirements (Temporal Autocorrelation):

Multiple wavelengths can be used to collect speckle data. Doing so allows for multi-parameter fitting, including tissue optical properties along with the blood flow index.

Multiple wavelengths may either be detected sequentially on the same detector or simultaneously with one wavelength per detector

### Aperture Requirements (Temporal Autocorrelation):

For speckle temporal autocorrelation measurements, in some cases no more than a “few” speckle should be detected simultaneously, ideally only one or possibly less than one speckle. Though more speckle may be collected for increased intensity with reduction in effective signal from reduction in coherence.

If a single mode fiber or waveguide is used to collect the light from tissue, then only one speckle is captured by definition of it being single mode.

Alternatively, the speckle size may be calculated without the use of imaging optics (e.g. no lenses) approximately by:

$$d = λ z / D$$

where d is the diameter of the speckle, λ is the wavelength, z is the distance from the aperture (or diameter of waveguide) to the detector, and D is the diameter of the aperture (or waveguide)

For example, for 1300 nm light and the desire to limit the distance from aperture to detector to 1 mm, and for speckle size to at least equal the detector size where the detector is 70 μm in diameter, the aperture size, D, should be no larger than D=1300e-9*1e-3/70e-6=18 μm. This aperture diameter is slightly larger than the core size of a single mode fiber owing to the way light is propagated partially in the fiber cladding. Either an aperture or a single-few-mode waveguide could be used to deliver light to the detector.

There is also the possibility of using imaging optics to deliver the light. In which case the speckle diameter is calculated as

$$d \approx 1.2 ( 1 + M ) λ f \#$$

$$where$$

$$NA = n sin ( arctan ( 1 / 2 f \# ) ) \approx 1 / 2 f \#$$

### Processing (Temporal Autocorrelation):

The correlation diffusion equation utilizes the diffusion approximation to provide an analytical solution to the complex electric field temporal correlation:

$${G}_{1} ( τ ) = \frac{{s}_{0}}{4 π D} [ \frac{exp ( - K ( τ ) {r}_{1} )}{{r}_{1}} - \frac{exp ( - K ( τ ) {r}_{2}}{{r}_{2}} ]$$

where τ is the delay time, S₀ is the source intensity, D is the optical diffusion coefficient with

$$D = \frac{1}{3 ( {μ}_{a} + {μ}_{s}^{′} )} , and K ( τ ) = \sqrt{3 {μ}_{a} {μ}_{s}^{′} + {μ}_{s}^{r 2} {k}_{o}^{2} 〈 Δ {r}^{2} ( τ ) 〉}$$

where k₀=2πn/λ with n the index of refraction and λ the wavelength of light and [symbol] Δr²(τ) [symbol] is the average mean-squared displacement. In tissue the approximation is made that flow is diffusive and thus [symbol] Δr²(τ) [symbol] =6D_{B}t where D_{B} is the effective Brownian diffusion coefficient of scattering particles and approximately related to blood flow index. Often, the G₁ equation is normalized by average intensity and is then written as g₁. The intensity temporal autocorrelation, g₂, is related to g₁ by the Siegert relation: g₂(τ)=1+βg₁(τ)² where β accounts for loss of correlation that may be due to laser coherence length, number of speckle, including ratio of detector size to speckle size, and polarization, among other factors. Typically, the temporal autocorrelation at evenly spaced lag times (linear correlation) or logarithmically spaced lag times (multi-tau correlation approach) up to about 1 ms is calculated to obtain the g₂ decorrelation curve. g₂ is then fit with a known equation to obtain g₁. g₁ is fit to obtain the blood flow index by using assumed optical properties or by using multiple wavelength data or multiple spatial data to obtain real tissue optical properties along with the blood flow index.

If only the relative blood flow is desired, rather than obtaining g₂ with fine resolution by calculating temporal autocorrelation at evenly spaced lag times, the sampling could be adjusted such that a minimum number of points are needed to fit g₂ to find the decorrelation time, tau. Further, in some embodiments, it may not be necessary to calculate or fit g₂ at all. Instead, the photonic integrated device may be configured to simply monitor the change in shape of the autocorrelation vs time via an algorithm.

### Practical Use Case (Temporal Autocorrelation):

Note that interferometric measurements of speckle temporal correlation, sometimes referred to as interferometric diffuse correlation spectroscopy (iDCS) may be preferred over speckle pattern measurements due to size constraints in a wearable format. Speckle contrast measurements require multiple pixels/detectors and therefore a larger area sensor. There are also limitations on detector size and distance from the tissue. Whereas iDCS only requires a single detector and has no constraint on detector size as long as an aperture or waveguide is utilized to deliver light.

However, it is possible to expand the example embodiments disclosed herein to include the use of multiple detectors for calculation of speckle contrast in addition to or rather than temporal autocorrelation. Where speckle contrast is utilized, the following modifications apply:

### Speckle Contrast as an Addition or an Alternative to Temporal Autocorrelation:

Measure blood pressure via SPG measurement using temporal change in speckle pattern in a wearable form factor using a PIC that includes the laser and detectors. Detectors may be discreet detectors, a detector array or an image sensor. Speckle contrast measurements are performed in a custom ASIC or programmable FPGA that is part of the full hardware stack.

### Mode of Operation (Speckle Contrast)

Continuous wave or pulsed. For pulsed operation, preferably the pulse duration should be either significantly longer than the speckle contrast measurement time (integration time) or significantly shorter than the speckle contrast measurement time.

### Data Sampling Rate (Speckle Contrast):

For speckle contrast measurements, data collection should be at least 20 Hz to capture the SPG waveform, though preferably at least 90 Hz, or at least 100 Hz.

The data sampling rate may be further increased to acquire at least 15 “frames” of data at 20 Hz, which equates to 300 Hz. This allows for a temporal calculation to be performed on the speckle contrast data, which improves accuracy/reduces error.

Discreet detectors or sets of linear arrays may be better suited to this task than high frame rate cameras, which are expensive due to the large number of pixels. This application would only require, e.g. an 8×8 array which could have a lower burden on readout design.

### Aperture Requirements (Speckle Contrast):

Speckle diameter (using calculations above) should be at least the same diameter as a given detector or pixel and preferably twice the size as a detector or pixel. Speckle diameter may be less than the size of a given detector or pixel, but will result in reduced signal quality.

### Detector Array (Speckle Contrast):

Ideally the detector size is as small as possible to reduce distance from the aperture to the detector (see above calculations for speckle size, diameter of aperture, and distance).

Literature teaches that a minimum of 5×5 pixels (detectors) and preferably 7×7 or more are used to calculate the speckle contrast, defined as

$${K}_{s} = \frac{{σ}_{s}}{〈 I 〉} .$$

Where σₛ is the spatial standard deviation of speckle intensity.

Specifically, literature teaches that relationship between variation of speckle contrast and window size is given by:

$${σ}_{g} = 1 + 0.4 \; 54 {p}^{0.672} {N}_{pixels}^{- 0.373}$$

$$where p = {ρ}_{speckle} / {ρ}_{pixel}$$

The discreet detectors in an array format do not need to be rectilinear but may be interdigitated or distributed in any pattern about the light source, symmetric or asymmetric. The total number of detectors should be at least 16, preferably at least 49. More detectors reduces the error in calculation of speckle contrast.

It is envisaged that one could combine temporal autocorrelation and speckle contrast in the same device for additional error reduction.

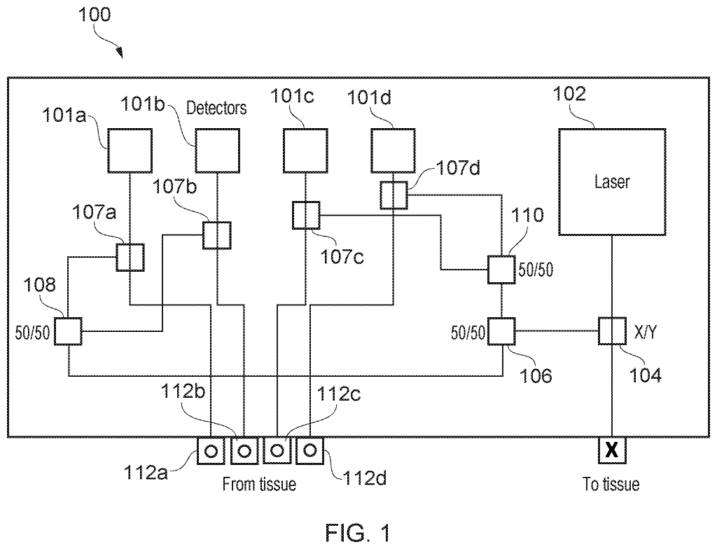

FIG. 1 shows a photonic integrated circuit 100, PIC capable of temporal SPG with 4 detectors, and heterodyne detection. The PIC 100 includes a laser 102, an output of which is provided to splitter 104, and four detectors 101a-101d. The exact ratio of the split (X/Y) may be variable. Whereas in most cases a 50% split is preferred, in this example, X takes a value of 10% and Y takes a value of 90%, where X represents the fraction of light provided to the sample arm (e.g. tissue) and Y represents the fraction of light provided to the local reference arm (e.g. local oscillator).

The light provided to the sample arm then passes through a second splitter 106, which divides the light into a first and second path at a ratio of 50:50. The first path connects to a third splitter 108, which again splits the received light at a ratio of 50:50 and provides each portion to a respective detector 101a, 101b. The second path connects to a fourth splitter 110, which again splits the received light at a ratio of 50:50 and provides each portion to a respective detector 101c, 101d. Each detector therefore receives ¼ of the light provided from the first splitter to the second splitter.

The light provided to the sample arm is then transmitted off of the chip to the tissue. The light is reflected from the sample, and a speckle grain is received into each of four receivers 112a-112d, corresponding (and connected to) one of the respective detectors. Combiners 107a, 107b, 107c, 107d are located before the detectors and act to combine the light that has been tapped from the laser with the light that has been reflected from the sample (e.g. skin) via the receiver and subsequent waveguide. A combiner could, for example, take the form of a 3 dB coupler (e.g. a 2×1 Y-junction or 2×1 MMI). The PIC in FIG. 1 forms a base architecture for PICs according to the present disclosure. Variable splitting ratios are particularly important in the field of biomedical applications, since it is undesirable to raise the power of light incident on tissue to levels that could cause damage to the tissue and/or discomfort to the user.

Although one laser is shown in FIG. 1, alternative embodiments include a plurality of lasers, for example a bank of lasers on a single PIC. Each laser may have a different wavelength or wavelength range to that of the others. The specific splitting ratios, firstly from laser into sample and reference arms and secondly from sample and reference arms into detector, may be calculated based on signal levels to maximize heterodyned SNR.

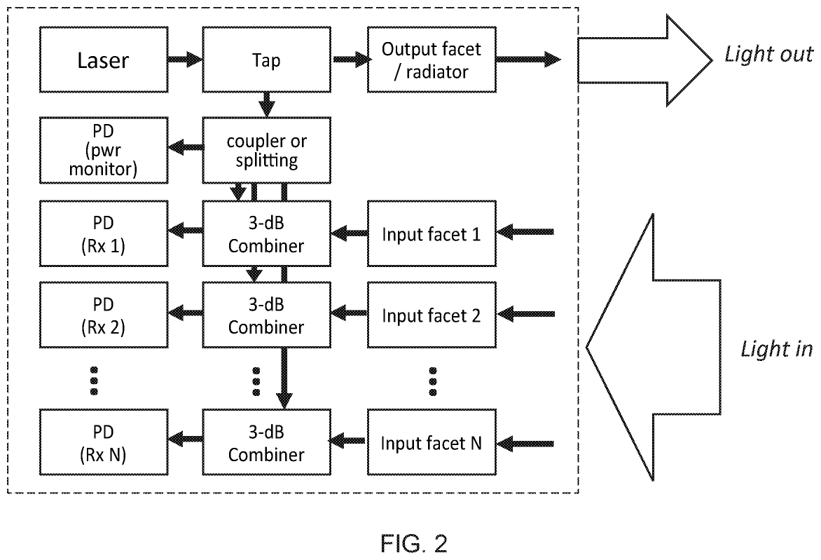

More details of this base architecture can be understood with reference to FIG. 2, which corresponds to a coherent example, showing more detail. The laser provides coherent light to a tap (e.g. corresponding to the splitter 104 shown in FIG. 1). A portion of the light is provided to an output facet/radiator so that it illuminates a sample. The remaining portion of the light (in this example 90% of the original light from the laser) is provided to a 1×N Star coupler or splitter tree. The star coupler or splitter tree provides one portion of the light to a photodiode which functions as a power monitor. The remaining light is provided to each of n combiners (in this example 3-dB combiners, other values are possible). In this example this is done equally, so that each of the n combiners receives light of a same or similar power level. The combiners each combine the light received from the star coupler or splitter tree with light received from a respective input facet (i.e. light which has reflected from the sample, and corresponds to a single grain in a speckle pattern). The combined light is then provided to a respective photodiode (Rx 1, Rx 2 . . . . Rx N).

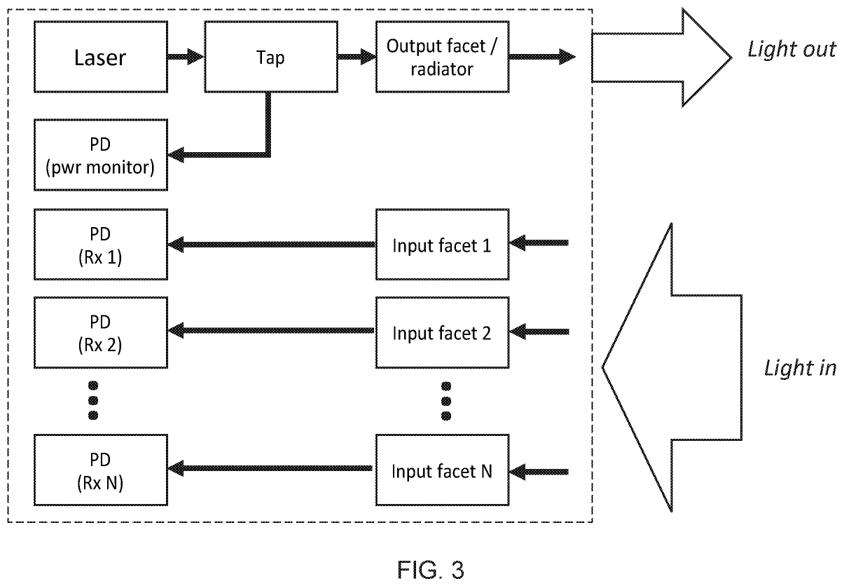

FIG. 3 shows an alternative base architecture, in the form of a direct detect example, where no reference arm is provided. Instead, the laser output is split by a tap, as before, but a portion is only provided to the photodiode functioning as a power monitor. All of the remaining light is used to illuminate the sample by provision to the output facet/radiator. Reflections of the laser light are received by each of the input facets, which are directly connected (without an intermediary combiner) to the respective photodiodes forming the detectors.

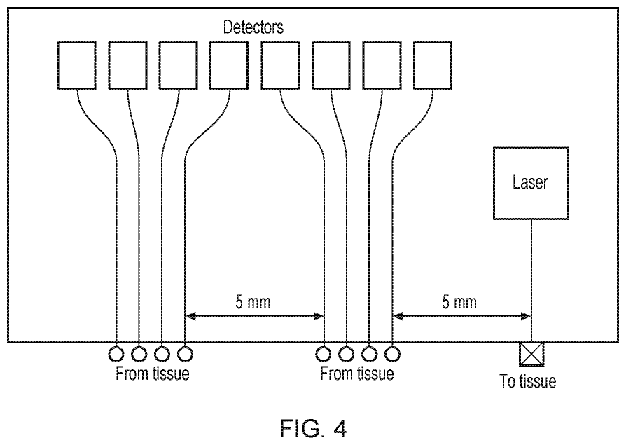

FIG. 4 shows a PIC which is operable as a temporal SPG, with two sets of four detectors. The first set of detectors (rightmost) collecting from light from a first position e.g. 5 mm from the output facet (the source of light), and the second set (leftmost) collecting light from a second position further away from the output facet than the first position, e.g 10 mm from the output facet.

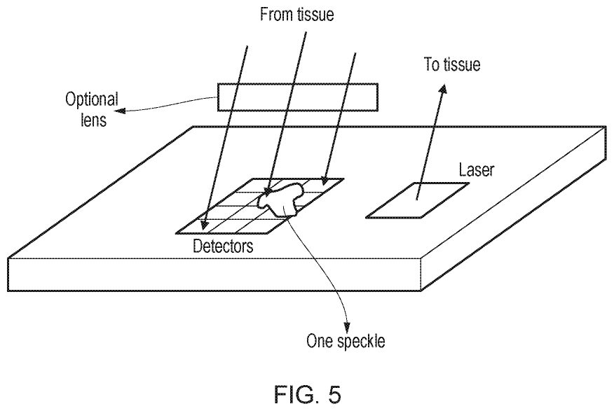

FIG. 5 shows a PIC which is operable as a spatial SPG, with a 4×4 array of closely spaced detectors. Other sized areas are possible, for example 5×5, 10×10, or 100×100. An optional lens is shown, for imaging light from the tissue surface onto the detectors. In some embodiments, the detectors in FIG. 5 have different sizes or dimensions. In some embodiments, some of the detectors are used for temporal SPG whilst the other detectors are used for spatial SPG. Specifically, in some examples the outermost detectors (e.g. those around the edge of the detector array) are used for temporal SPG whilst the inner detectors are used for spatial SPG.

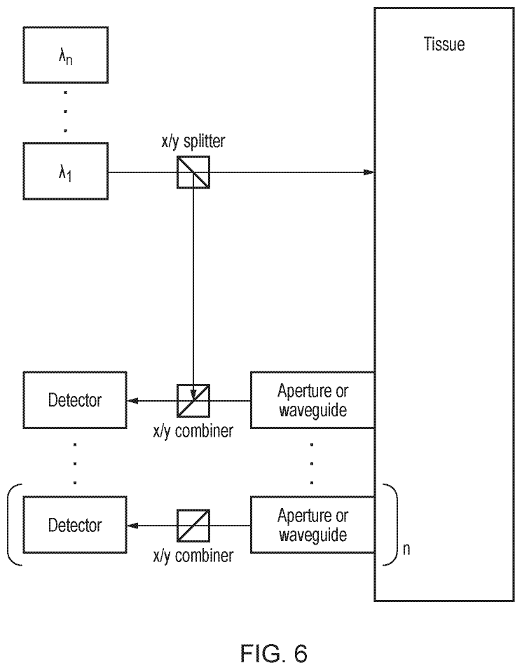

FIG. 6 is a schematic diagram of a PIC according to an embodiment of the present invention in use. A single source position is used (that is, light is emitted from a single facet/radiator on an end of the PIC). In this example a single wavelength, λ₁, of coherent light is provided. But multiple wavelengths, λₙ, can be used. If so, the light is combined before emission through the single source. The PIC also includes at least one (but may include more) detectors. In this example the light provided from the or each laser is split at a ratio of X/Y by a splitter located between laser(s) and the output facet. Where multiple wavelengths are used, the combination of those wavelengths is carried out before the X/Y splitter. Part of the laser light is provided to the sample via the output facet, to illuminate the sample, and part of the laser light is provided to a combiner located between the or each input facet (which can be an aperture or a waveguide) and the respective detector.

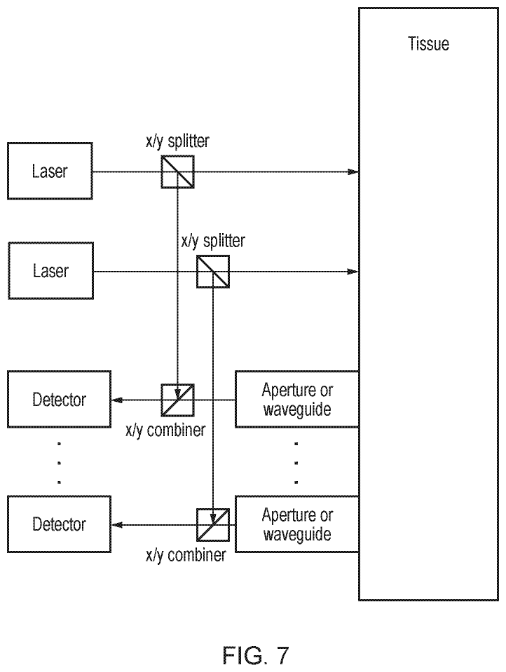

FIG. 7 is a schematic diagram of a PIC according to an embodiment of the present invention in use. Multiple source positions are used, in that light is emitted from a plurality of output facets/radiators on an end of the PIC. This may be achieved through a single laser, coherent light from which is split before provision to each output facet/radiator, or through a laser for each of the output facets/radiators. As before, a portion of the light to be outputted through the output facets/radiators is split via a splitter and provided to a combiner located between each input facet (which can be an aperture or a waveguide) and the respective detector.

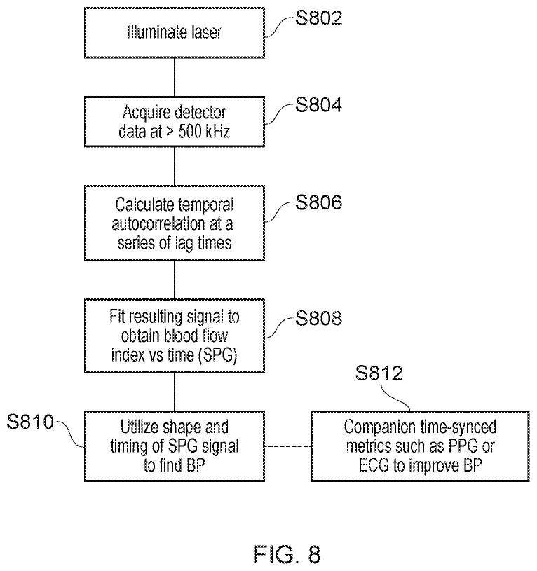

FIG. 8 is an example of a method of use of a PIC according to an embodiment of the present invention. In a first step, S802, the laser is illuminated (switched on) and so coherent light is incident on a sample. Next, in step S802, detector data is acquired at a rate in excess of 500 kHz. From this, in step S806, temporal autocorrelation is calculated at a series of lag times. The resulting signal is then fitted to obtain a blood flow index v time (SPG) in step S808. Properties of the SPG signal including shape and timing are then utilized to determine blood pressure in step S10, this determination can utilize companion time-synced metrics such as a PPG or ECG to improve the accuracy with which BP is derived.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention. All references referred to above are hereby incorporated by reference.

What is claimed is:

1. A device adapted to investigate blood flow at a portion of tissue of a user, the device comprising:
a laser configured to generate light for transmission to the portion of the tissue;
a plurality of detectors; and
a plurality of separate optical inputs respectively optically coupled to the plurality of detectors and configured to respectively receive a plurality of separate speckle patterns, responsive to the transmitted light, from the portion of the tissue,
wherein the device is configured to obtain a speckleplethysmography (SPG) measurement based on a plurality of measurements taken respectively by the plurality of detectors.

2. The device of claim 1, wherein the laser and the plurality of detectors are located on a first photonic integrated chip.

3. The device of claim 1, wherein the laser is located on a first photonic integrated chip (PIC), and the plurality of detectors are located on a second PIC separate from the first PIC.

4. The device of claim 1, further comprising an ASIC or FPGA, said ASIC or FPGA configured to calculate temporal autocorrelation of a detected intensity.

5. The device of claim 4, wherein the ASIC or FPGA is configured to carry out speckle contrast measurements between measurements obtained by different detectors of the plurality of detectors.

6. The device according to claim 1, further comprising a single mode waveguide or an aperture adapted to receive light from the portion of the tissue of the user, said single mode waveguide or aperture placed in optical communication with a respective one of the plurality of detectors.

7. The device according to claim 1, further comprising a multimode waveguide adapted to receive light from the portion of the tissue of the user, said multimode waveguide placed in optical communication with a respective one of the plurality of detectors.

8. The device of claim 1, wherein the device is configured such that each of the plurality of detectors receives only light from a single grain of speckle within the corresponding speckle pattern.

9. The device of any claim 1, wherein the laser is located on a first photonic integrated circuit (PIC), the first PIC being a silicon photonics chip.

10. The device of claim 1, wherein the laser has a wavelength of operation of 1280 nm or more.

11. The device of claim 1, wherein the laser is a fixed wavelength laser.

12. The device of claim 1, wherein the laser is a tunable laser.

13. The device of claim 1, comprising a plurality of lasers, including the laser, located on a photonic integrated circuit.

14. The device of claim 13, wherein each laser of the plurality of lasers has a different wavelength or wavelength range of operation.

15. The device of claim 13, wherein the plurality of lasers are configured to be operated one laser at a time.

16. The device of claim 1, wherein the device is configured to measure blood pressure via the SPG measurement.

17. The device of claim 1, wherein the SPG measurement is based on a spatial standard deviation of speckle intensity of the plurality of measurements taken by the plurality of detectors.

18. The device of claim 1, wherein each of the plurality of separate optical inputs is configured to receive only approximately one speckle grain within the corresponding speckle pattern.

19. The device of claim 1, wherein the device is configured such that each of the plurality of detectors measures less than five speckle grains within the corresponding speckle pattern.
